(19) United States
(12) Patent Application Publication
(10) Pub. No.: US 2005/0208637 A1
(43) Pub. Date: Sep. 22, 2005

(54) Membraine filtration module and method for the separation of biomolecules from a liquid

(75) Inventors: Wolfgang Demmer, Gottingen (DE); Dietmar Nussbaumer, Gottingen (DE); Hans-Heinrich Horl, Bovenden (DE)

Correspondence Address: Chernoff Vilhauer;McClung & Stenzel, 1600 Ods Tower, 601SW Second Avenue, Portland, OR, 97204-3157, US

(73) Assignee: Sartorius AG

(21) Appl. No.: 10/516,404

(22) PCT Filed: Jun. 21, 2003
(86) PCT No.: PCT/EP03/06564

§ 371 Date: Nov. 30, 2004

(30) Foreign Application Priority Data
Jul. 23, 2002 (DE) 102 33 542.7

(51) Int. Cl. C12N 9/00; B01D 39/14

(52) U.S. Cl. 435/183; 210/500.21

(57) ABSTRACT

The invention describes a membrane and a method of making and using the same, the membrane being made from a microporous membrane body with an affinity ligand capable of interacting with at least one type of biomolecule found in a fluid, whereby the membrane body may be stored in a dried state yet retain the activity of the affinity ligand.

This is a 371 of PCT/EP 2003/006564 filed 21 Jun. 2003 and claims priority of DE 102 33 542.7 filed 23 Jul. 2002.

### BACKGROUND OF THE INVENTION

Spherical carriers in the form of gels containing affinity ligands have been employed for a long time in many areas of biotechnology for purification and separation of many different types of biomolecules. An example of such an application is the use of affinity ligands based on an agarose gel, commercially available in matrices such as aqueous suspensions or in lyophilized form. A persistent problem with such matrices containing affinity ligands is that it is difficult if not impossible to dry the matrices once they have become swollen in an aqueous medium because the small gel spheres are irreversibly damaged during drying. Preservation and transport of such gels thus presents a considerable logistical problem.

From EP 0 787 523 A1 it is known that ligands of a carrier material can be coupled for separation of substances having an affinity for the ligands. The function of the ligands is to bind a single target substance or even an entire class of substances which specifically adsorb to the ligands. It is further known from DE 196 17 775 A1 that membrane adsorbers or membranes can carry ligands which are capable of interacting with at least one substance in a liquid phase. The transport of the liquid phase through the membrane occurs in this case convectively due to a pressure differential. A chief drawback of such known separations of biomolecules is that the presence of water moisture in the carrier or membrane involves the risk of microbial attack, thereby requiring the addition of preservatives. But at the same time, the carriers or membranes tend to dry out, which drying must be suppressed with complicated procedures in order to prevent loss of the bioactivity of the ligands.

A primary object of the present invention is therefore to provide membranes for separation of biomolecules from a fluid by means of affinity ligands that permits elimination of complicated and costly wet storage of the membranes.

### BRIEF SUMMARY OF THE INVENTION

The foregoing object is achieved by the present invention, which allows dry storage of the membrane with the affinity ligand, yet retains the activity of the affinity ligand.

Because the membrane can be stored practically without a significant loss of activity, storage and transportation costs can be significantly reduced and the separation of biomolecules is simplified.

There are essentially three aspects to the invention: (1) a microporous membrane infused with an affinity ligand capable of interacting with at least one type of biomolecule in a fluid; (2) a filtration module for the separation of biomolecules from a fluid, comprising a housing and at least one membrane of the type noted in (1); and (3) a method for the separation of biomolecules from a fluid by one or more membranes of the type noted in (1) or by a filtration module of the type noted in (2).

### BRIEF DESCRIPTION OF THE DRAWINGS

FIG. 1 is a schematic of an exemplary filtration module of the invention with a single membrane in a housing.

FIG. 2 is a schematic of an exemplary filtration module of the invention with several membranes arranged in a series in a housing.

FIG. 3 a schematic of an exemplary filtration module of the invention with membranes arranged in several layers in a housing.

### DETAILED DESCRIPTION OF PREFERRED EMBODIMENTS

Surprisingly, it has been found that membranes charged with affinity ligands such as proteins can be stored dry for a relatively long period of time without a loss of activity. A particularly suitable class of microporous membranes of this type is that which is commercially available from Sartorius AG of Göttingen, Germany under the trade name of Sartobind®. The term “dry” as used herein should be understood as relating to membranes or membrane bodies whose pore volume is substantially filled with air. This does not exclude those cases where the inner surface of the pores is covered with a highly volatile organic substance.

Suitable membranes are polymeric microporous membranes such as cellulose acetate (CA), cellulose nitrate (CN), polyamide, polyether sulfone (PES), polypropylene (PP) and polyvinylidine fluoride (PVDF). The diameter of the pores for such membranes should be from 0.01 to 15 μm, preferably from 0.2 to 5 μm. The thickness of such membranes is from 100 to 500 μm, preferably from 200 to 300 μm. Such membranes are preferably chemically activated, so that the affinity ligands can be chemically coupled thereto. However, physical binding of the affinity ligands to the membranes is also possible. In a preferred embodiment, the membrane of the invention is impregnated with glycerine, which aids in preventing damage to the membrane's microporous structure during the drying process.

Adsorptive affinity ligands are well known to persons skilled in the art, and include the following:
thiophiles;
hydrophobes with various chain lengths and configurations;
reversed phase ligands;
dyes, including reactive dyes;
low molecular weight charged or non-charged organic molecules;
amino acids and analogs thereof;
coenzymes, cofactors and analogs thereof;
substrates and analogs thereof;
endocrine and exocrine substances such as hormones and substances having an effect similar to that of hormones and analogs thereof;
enzyme substrates, enzyme inhibitors and analogs thereof;
fatty acids, fatty acid derivatives, conjugated fatty acids and analogs thereof;
nucleic acids, including DNA, RNA and analogs and derivatives thereof;
monomers and analogs and derivatives thereof;
polymers and oligopolymers and analogs and derivatives thereof;
high molecular weight carbohydrates, linear or branched chain and substituted or unsubstituted;
glycolic conjugates, such as
   heparin;
   amylose, cellulose;
   chitin, chitosan;
   lignin;
   and monomers, oligomers, and derivatives and analogs thereof;
high molecular weight ligands such as
   proteins and oligomers, subunits and parts thereof;
   peptides, polypeptides and analogs and derivatives thereof;
   lectine;
   antibodies and parts thereof;
   fusion proteins; and
   haptenes;
enzymes and subunits and parts thereof;
structural proteins;
receptors and parts thereof;
xenobiotics;
pharmaceuticals and pharmaceutically active substances;
alkaloids;
antibiotics; and
biomimetic substances.

In another preferred embodiment of the invention, selective separation of different biomolecules can be achieved by using a plurality of membranes having affinity ligands coupled thereto. Moreover, the types of membranes can be adjusted in a relatively simple manner, depending on the relevant separation problem. The membranes can be arranged in a housing in multiple layers and can also be arranged serially in single housing chambers or in different housings.

Another aspect of the invention is to provide an efficient and cost-effective method for the membrane separation of biomolecules from a fluid, which is possible without the need for complicated wet storage and transport of the membranes used. The method comprises the following steps:
(a) coupling at least one affinity ligand to the separation membrane in a solution;
(b) washing the separation membrane of step (a) with at least one washing medium;
(c) removing the washing medium of step (b) by drying;
(d) dry storage of the dried separation membrane of step (c); and
(e) filtering a fluid containing biomolecules through the dried separation membranes, so as to separate the biomolecules.

To minimize the risk of microbial attack when water is used as the washing medium, the separation membrane is preferably dried to a water activity of about 40%. As used herein, the term “water activity” means the equilibrium partial pressure of water in the membrane relative to pure water at the same temperature. In step (b) above a strongly volatile organic substance of one or more components that are miscible with the washing medium may be added as an impregnation medium which remains in the membrane during the drying stage. A film can be also formed on the surface of the pores or the membrane can be formed in a swollen state.

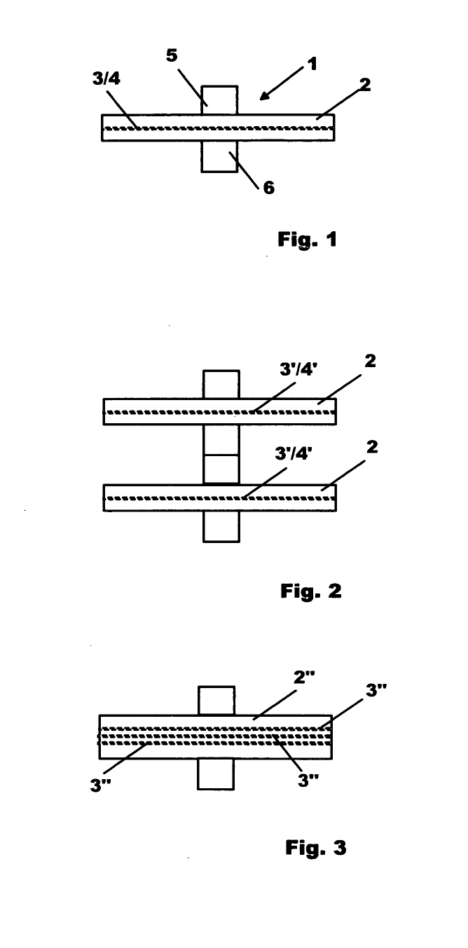

Referring to the drawings, wherein like numerals refer to the same elements, there is shown in FIG. 1 a filter module 1 for the separation of biomolecules from a fluid essentially comprising a housing 2, a membrane 3 having a membrane body 4, an inlet 5 and an outlet 6. Membrane body 4 is microporous and adsorptive and may be made from CA, CN, polyamide, PES, PP or PVDF with an average pore diameter of from 0.01 to 15 μm, preferably 0.2 to 5 μm. Membrane body 4 is preferably planar and has a thickness from 100 to 500 μm, more preferably from 200 to 300 μm. Affinity ligands of the types previously described are coupled to membrane body 4, and are selected so that they have the capability to interact with the biomolecules to be separated from the processed liquid.

Although membrane body 4 can be provided as a single layer arranged in housing 2 as shown in FIG. 1, multiple housings, each provided with a membrane body 4′ can be arranged in series, as shown in FIG. 2.

It is also possible to arrange membranes 3″/membrane bodies 4″ in several layers in one housing 2″, as shown in FIG. 3. Membrane bodies are preferably impregnated with glycerine and then subjected to drying, so as to remove water to a very high degree. After dry storage or after transport, the fluid containing biomolecule(s) for processing is supplied through inlet 5 and transported convectively through the membranes, thereby binding the biomolecules to be separated to the affinity ligands. Filtered fluid is discharged through outlet 6.

### EXAMPLE

A phosphate buffered saline (PBS) solution having a pH of 7.3±0.2 was prepared as described by J. Sambrook et al. in “Molecular Cloning—A Laboratory Manual,” Book 3, Appendix b. 12 (2d ed. 1989) by combining the following components in the concentrations noted in an aqueous solution.

| Concentration (g/L) | Substance |
|---|---|
| 8.00 | NaCl |
| 0.20 | KCl |
| 1.44 | Na₂HPO₄ |
| 0.24 | KH₂PO₄ |

Three 25 mm disks of a regenerated cellulose microporous membrane functionalized with aldehyde groups (Sartobind® Aldehyde Membrane Code 19306) were reacted with Protein A which contains primary amine groups (Repligen Company, Designation rPrA, Lot No. 011038) to form a Protein A affinity ligand coupled via aldehyde/amine chemical links to the membranes. The protein was dissolved in 10 mg/mL of the PBS solution. The three membrane disks were placed in a Petri dish with 2 mL of the Protein A/PBS solution and agitated for three hours at ambient temperature to form reversible Schiff bases from the aldehyde/amine links. The so-formed Schiff bases were reduced by the addition of 10 mg/mL sodium cyanoborohydrate. After the reaction took place, the membranes were removed and transferred to a fresh Petri dish. In order to reduce the remaining aldehyde groups, 5 mL of a solution of sodium borohydride in PBS with a final concentration of 1% was added to the membranes and the same were agitated for another 15 minutes. The membranes were then washed sequentially with the following solutions: PBS; 0.1 M glycine, adjusted to pH 2.7 with HCl; 1 mM HCl in water; 1 mM NaOH in water; and 1 mM NaCl in 0.01 M potassium phosphate, pH 7.0. The membranes were then dried at ambient temperature with an air current for 3 hours and stored at 4° C. while air was substantially excluded.

The membranes were removed from storage after the storage times noted in the table below and tested with respect to their binding capacity for human immunoglobulin of the type IgGlund IgG2. Filtration modules for the tests were made by incorporating three of the membranes described above into a syringe adaptor unit with a diameter of 25 mm and equipped with a disposable syringe (both from Sartorius AG).

Processed human plasma from a local blood bank was diluted with PBS to a ratio of 1:40 and this solution was first filtered through a 0.2 μm membrane. The syringe was filled with 10 mL of the pre-filtered solution and gravity filtration with the Protein A-coupled membranes was carried out. Following this filtration, washing was conducted with 10 mL of PBS and the bound IgG was eluted with 10 mL of 0.1 M glycine, pH 2.7. The absorption of the elution solution was determined at 280 nm with a spectrum photometer and a manually adjustable calibration apparatus using bovine serum albumin (BSA) as a control to determine the protein-binding capacity. The IgG binding capacity of the functionalized membranes for Protein A as a function of time for these tests are shown in the table below. All values were median values obtained with at least 2 measurements.

| Time (Days) | Binding Capacity (μg/cm²) |
|---|---|
| 0 | 42 |
| 1 | 41 |
| 4 | 47 |
| 20 | 43 |
| 45 | 40 |
| 56 | 37 |

The terms and expressions which have been employed in the foregoing specification are used therein as terms of description and not of limitation, and there is no intention in the use of such terms and expressions of excluding equivalents of the features shown and described or portions thereof, it being recognized that the scope of the invention is defined and limited only by the claims which follow.

1. A method of preparing a membrane having an affinity for biomolecules comprising the steps:
(a) providing a microporous membrane;
(b) reacting said membrane of step (a) with a reagent containing a functional group to form a functionalized membrane containing reactive functional groups on the surfaces thereof;
(c) contacting said functionalized membrane of step (b) with a solution containing an affinity ligand to couple said ligand to said functional group to form a biologically active membrane;
(d) washing said biologically active membrane with a washing solution containing a volatile organic compound that is miscible with said washing solution; and
(e) drying said biologically active membrane.

2. The method of claim 1 wherein said functional group of step (b) is an aldehyde.

3. The method of claim 1 wherein said affinity ligand of step (c) is selected from the group consisting of thiophiles; hydrophobes; reversed phase ligands; dyes; low molecular weight charged or non-charged organic molecules; amino acids and analogs thereof; coenzymes, cofactors and analogs thereof; substrates and analogs thereof; endocrine and exocrine substances; enzyme substrates, enzyme inhibitors and analogs thereof; fatty acids, fatty acid derivatives, conjugated fatty acids and analogs thereof; nucleic acids; monomers and analogs and derivatives thereof; polymers and oligopolymers and analogs and derivatives thereof; high molecular weight carbohydrates; glycolic conjugates; proteins and oligomers, subunits and parts thereof; peptides, polypeptides and analogs and derivatives thereof; lectine; antibodies and parts thereof; fusion proteins; haptenes; enzymes and subunits and parts thereof; structural proteins; receptors and parts thereof; xenobiotics; pharmaceuticals and pharmaceutically active substances; alkaloids; antibiotics; and biomimmicking substances.

4. The method of claim 3 wherein said affinity ligand is Protein A.

5. The method of claim 1 wherein said washing solution of step (d) is aqueous-based.

6. The method of claim 5 wherein said washing solution is a phosphate buffered saline solution.

7. The method of claim 6 wherein said volatile organic compound of step (c) is glycerine.

8. The method of claim 1 wherein step (c) is conducted at ambient temperature.

9. The method of claim 1 wherein said microporous membrane of step (a) is a polymeric membrane selected from the group consisting of cellulose acetate, cellulose nitrate, polyamide, polyethersulfone, polypropylene and polyvinylidene fluoride.

10. The method of claim 9 wherein said microporous membrane has an average pore diameter of from about 0.01 to about 15 microns and a thickness of from about 100 to about 500 microns.

11. The membrane product of the method of any of claims 1-4.

12. The membrane product of claim 11 stored in a dry state in a substantially anaerobic atmosphere.

13. At least one of the membrane product of claim 11 in a filtration housing having a fluid inlet and a fluid outlet wherein said at least one membrane product is situated between said inlet and said outlet.

14. The membrane product of claim 13 wherein said filtration housing contains three of said membrane products.
